(19) United States
(12) Patent Application Publication
(10) Pub. No.: US 2023/0181277 A1
(43) Pub. Date: Jun. 15, 2023

(54) SYSTEM FOR CONNECTING MEDICAL DRAPING

(71) Applicant: Microtek Medical, Inc., Columbus, MS (US)

(72) Inventors: Kenneth James Klann, Willernie, MN (US); Joshua David Gunn, Marietta, GA (US); Daniel Wald, Burnsville, MN (US)

(21) Appl. No.: 18/066,168

(22) Filed: Dec. 14, 2022

Related U.S. Application Data
(60) Provisional application No. 63/289,548, filed on Dec. 14, 2021.

(51) Int. Cl. A61B 46/10; A61B 46/23; A61B 46/00

(52) U.S. Cl. CPC A61B 46/10; A61B 46/23; A61B 46/40

(57) ABSTRACT

A drape connector system can be used to join different sections of drape material, such as different sections of compositionally dissimilar drape material. In some examples, a medical drape is formed that includes a first section of drape material, a second section of drape material, and the connector system. The connector system includes a receiving body defining a groove and a locking body insertable into the groove. A portion of the first section of drape material is positioned overlaying the groove, and a portion of the second section of drape material is also positioned overlaying the groove. The locking body is inserted into the groove thereby locking the portion of the first section of drape material and the portion of the second section of drape material in the groove.

### RELATED MATTERS

This application claims the benefit of U.S. Provisional Patent Application No. 63/289,548, filed Dec. 14, 2021, the entire contents of which are incorporated herein by reference.

### TECHNICAL FIELD

This disclosure relates to drapes used in medical procedures, including drapes used to provide a sterile barrier over medical equipment during a surgical procedure.

### BACKGROUND

Medical drapes, also referred to as surgical drapes, can be used during surgical procedures to help protect patients and equipment from contamination. For example, a patient drape may be used to cover portions of a patient's body not involved in a surgical procedure, providing a window or segregated space for accessing a region of the patient's body on which the surgical procedure is being performed. As another example, an equipment drape can be used to cover portions of a piece of medical equipment used during a procedure. Example types of equipment that can be draped include imaging systems (e.g., C-arm imaging machines) and surgical equipment (e.g., robotic surgical systems). In either case, a drape can provide a physical barrier helping to protect the surgical field from contamination, protecting the patient, clinicians, and/or equipment. The appropriate use of medical drapes can help minimize the possibility of cross-contamination and hospital acquired infections between procedures and patients.

Surgical drapes can be made of a variety of different materials, including plastic, cloth, and paper. Surgical drapes can be designed for cleaning and reuse although more commonly are provided as a single-use disposable item. Each surgical drape used in a surgical environment may be designed for a specific purpose (e.g., covering certain patient anatomy, covering a specific piece of equipment). Accordingly, a surgical drape may be fabricated from a variety of different materials and/or have one or more accessory features connected thereto to configure the drape for its intended purpose. Individual drape components may typically be joined together using adhesive, heat welding, or the like. When working with drape components that have incompatibilities with each other (e.g., chemical incompatibility challenging adhesive bonding, thermal incompatibility challenging heat welding), there is an increased risk of subsequent joint failure between the components. If a surgical drape exhibits a joint failure during a medical procedure, there is risk of patient contamination from the environment, environmental/equipment contamination from the patient, and potential biohazard remediation requirements.

### SUMMARY

In general, this disclosure is directed to a sterile or non-sterile drape for use in a medical procedure that may include two or more drape components joined together through a connector system and associated methods and systems involving such drapes. In some examples, a medical drape (also referred to herein as a surgical drape) is formed of at least two physically separate sections of drape material that are joined together by the connector system. Each of the different sections of drape material may have a different composition, making fluid-tight connection between the different drape materials challenging. In other applications, however, the different sections of drape material can have the same composition. In either case, the connecting system can be used to physically interlock the different sections of drape material together, e.g., forming a joint that is fluid tight and is effective at resisting separation or other joint failure during expected use of the drape in a medical procedure.

In some implementations, the connector system includes two components: a receiving body defining a groove and a locking body insertable into the groove. The locking body can be configured (e.g., sized and/or shaped) to be retained in the groove once inserted therein, resisting pull out or separation of the locking body from the groove. In use, each section of drape material can be positioned overlaying the groove and the locking body inserted into the groove. After insertion, each section of drape material can be sandwiched in the groove between the receiving body and the locking body. The connection between the receiving body and locking body can be sufficiently tight to prevent movement of the drape sections trapped between the receiving body and locking body. For example, the locking body may be interference fit (also referred to as press fit or friction fit) into the receiving body. The connector system can be used to connect different sections of drape material without using chemical or thermal bonding between different sections of drape material at the joint line.

A drape formed from at least two sections of drape material connected using a connection system according to the disclosure can have a variety of different configurations. In general, the drape can be fabricated to have any desired configuration and satisfy any intended purpose within the surgical environment. The drape may be designed as a patient drape used to cover a portion of a patient during a surgical procedure. The drape may be designed as an equipment drape used to cover a portion of a piece of equipment used during the surgical procedure. The drape can be configured (e.g., sized, shaped) based on the portions of the patient's anatomy the drape is intended to cover and/or the configuration of the equipment the drape is intended to cover.

In some implementations, the drape is a flat drape intended to cover generally flat surfaces. In these implementations, the connector system may extend linearly across a planar face of the drape (partially or fully across the face), e.g., joining two generally flat sections of drape material together. In other implementations, the drape may define a cavity, such as a tunnel, designed to be positioned surrounding a structure to be draped. In these applications, the connector system may define a frame having a perimeter surrounding (e.g., matching) a perimeter of the cavity formed by the drape. For example, the connector system may include a receiving body that defines an annulus. Two sections of drape material each defining a tunnel can be positioned over a groove in the annulus and a circular locking body inserted into the groove. Once assembled, the connector system can form a joint between the tunnel regions of two sections of drape material while also providing an open core through which a structure to be draped can be inserted. In addition to forming a robust joint, the connector system may provide a convenient location for a user to grasp or manipulate the drape when installing over a structure to be draped.

A drape connector system according to the disclosure can be used for a variety of types of drape connection arrangements other than joining different sections of drape material together. As one example, the connector system can be used to connect a drape directly to a piece of surgical equipment to be draped. In this example, the surgical equipment can include one portion of the connector system (e.g., the receiving body, the locking body) integrated with or otherwise attached to the surgical equipment. The surgical drape can include the other portion of the connector system (e.g., the receiving body, the locking body). The drape can be positioned over the portion of the surgical equipment desired to be draped, and the portion of the connector system carried by the drape engaged with the portion of the connector system carried by the surgical equipment to physically connect the drape to the surgical equipment.

As another example, the connector system can be used to connect an accessory feature to a drape. In practice, a drape may be configured with a variety of different types of accessory features, such as a fluid port for delivering fluid through the drape (e.g., via a fluid line inserted through the fluid port), an electrical port for connecting equipment used under the drape (e.g., on or in the patient), and the like. The joint between the accessory feature and surrounding drape may present an increased risk of seal failure, particularly in instances where force is applied between the accessory feature and external device connected thereto.

In some examples of the present disclosure, a connector system is used to attach the accessory feature to the drape. The drape can have an opening defining a perimeter, and the drape accessory feature can also define a perimeter. At least a portion of the perimeter of the drape opening can be positioned overlaying the groove of a receiving body of the connector system. At least a portion of the perimeter of the drape accessory feature can be positioned overlaying the groove of the receiving body. Alternatively, the receiving body groove may be integrally formed with or connected to the accessory feature. In either case, the locking body can be inserted into the groove thereby locking the drape accessory in the opening to form a joint between the drape and accessory.

In one example, a medical drape is described that includes a first section of drape material, a second section of drape material, and a connector system for joining the first section of drape material to the second section of drape material. The connector system includes a receiving body defining a groove and a locking body insertable into the groove. According to the example, a portion of the first section of drape material is positioned overlaying the groove, and a portion of the second section of drape material is also positioned overlaying the groove. The locking body is inserted into the groove thereby locking the portion of the first section of drape material and the portion of the second section of drape material in the groove.

In another example, a method of attaching sections of a drape material together is described. The method includes positioning a portion of a first section of drape material in a groove defined by a receiving body of a connector system and positioning a portion of a second section of drape material in the groove defined by the receiving body of a connector system. The example specifies that positioning the portion of the second section of drape material in the groove may involve positioning the portion of the second section of drape material over the portion of the first section of drape material in the groove. The example method further includes inserting a locking body in the groove, thereby joining the first section of drape material and the second section of drape material.

In another example, a draped surgical robot is described. The draped surgical robot includes a surgical robot defining an arm having an end and a first section of drape material defining an enclosure positioned over the end of the arm of the surgical robot and a tunnel extending along a first portion of the arm away from the enclosure. The example also includes a second section of drape material defining a tunnel extending along a second portion of the arm from the first section of drape material, and a connector system joining the tunnel of the first section of drape material to the tunnel of the second section of drape material. The example specifies that the connector system includes a receiving body and a locking body. The receiving body defines a frame extending about a perimeter of the tunnel of the first section of drape material and the tunnel of the second section of drape material, the frame providing an open lumen. According to the example, the locking body is inserted into a groove of the frame, thereby trapping a portion of the first section of drape material and a portion of the second section of drape material between the groove and the locking body to form a joint between the first section of drape material and the second section of drape material.

In another example, a medical drape with accessory attachment is described. The medical drape includes a section of drape material having an opening formed therein, the opening defining a perimeter, and a drape accessory defining a perimeter. The medical drape also includes a connector system for joining the drape accessory to the section of drape material. The connector system includes a receiving body defining a groove and a locking body insertable into the groove. The example specifies that at least a portion of the perimeter of the drape defining the opening is positioned overlaying the groove, at least a portion of the perimeter of the drape accessory is positioned overlaying the groove, and the locking body is inserted into the groove thereby locking the drape accessory in the opening.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is an illustration of an example medical drape according to the disclosure.

FIG. 2 is an illustration of an example connector system that can be used on the drape of FIG. 1.

FIGS. 3A and 3B are illustrations showing an example interlocking arrangement of the connector system of FIG. 2.

FIGS. 3C and 3D are illustrations showing another example interlocking arrangement of the connector system of FIG. 2.

FIG. 4 is a perspective view of an example configuration of a connector system configured as an annular connector.

FIG. 5 is a perspective view of another example configuration of a connector system where a groove is provided on an axial face of a perimeter frame.

FIGS. 6A and 6B are illustrations showing an example interlocking arrangement of a connector system utilizing a groove with straight sidewalls.

FIGS. 7A and 7B illustrate an alternative configuration of a connector system where the sidewalls of the groove are angled relative a bottom wall.

FIGS. 8A-8F are sectional views of a receiving body illustrating different example configurations that may be defined by the receiving cavity of the receiving body.

FIGS. 9A and 9B are sectional illustrations of an example configuration of a receiving body and locking body pair illustrating an example complementary configuration.

FIGS. 10A and 10B are sectional illustrations of another example configuration of a receiving body and locking body illustrating an example complementary configuration.

FIGS. 11A-11E are sectional views of a locking body illustrating example cross-sectional shapes for the locking body.

FIG. 12 is a side view of an example configuration of a connector system in which a locking body is configured to expand after being inserted into a receiving body.

FIGS. 13A and 13B are exploded perspective and side views, respectively, of an example configuration of a drape illustrating an example configuration of a connector system connecting an accessory feature to the drape.

FIG. 14 is a flow diagram of an example method of attaching sections of drape material together using a connector system according to the disclosure.

### DETAILED DESCRIPTION

This disclosure is generally directed to surgical drapes utilizing a connector system to attach different features of the drape together, such as different sections of drape material together, or to attach the full drape itself to a non-drape structure, such as a piece of surgical equipment to be draped. The connector system can include a receiving body and a locking body, with the locking body being insertable into the receiving body to lock the two components together. Portions of the different drape features desired to be joined together can be positioned between the locking body and the receiving body before the locking body is inserted into the receiving body. Once the locking body is subsequently inserted into the receiving body, an interlocked connection may be formed sandwiching the portions of the different drape features together and forming an integral joint between the different drape features. The joint may be continuous or discontinuous across the different drape features and, in some examples, may form a fluid-impermeable seal across the joint created by the connector system.

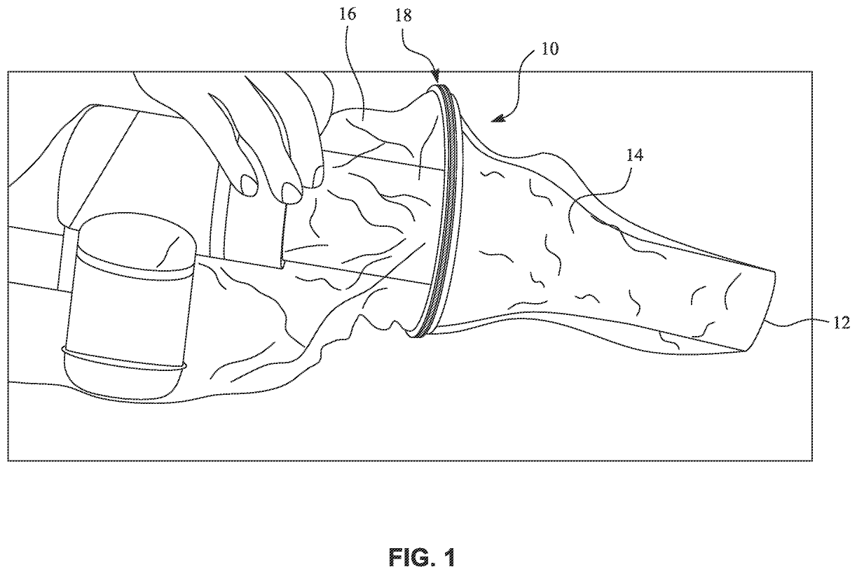

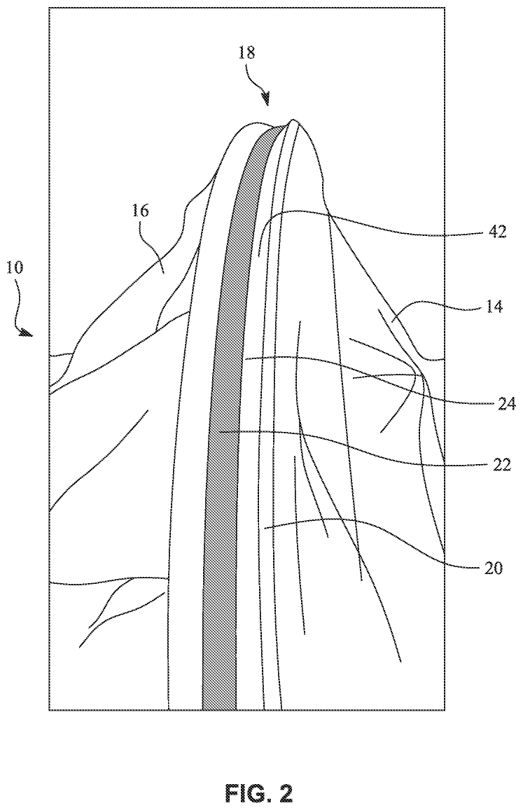

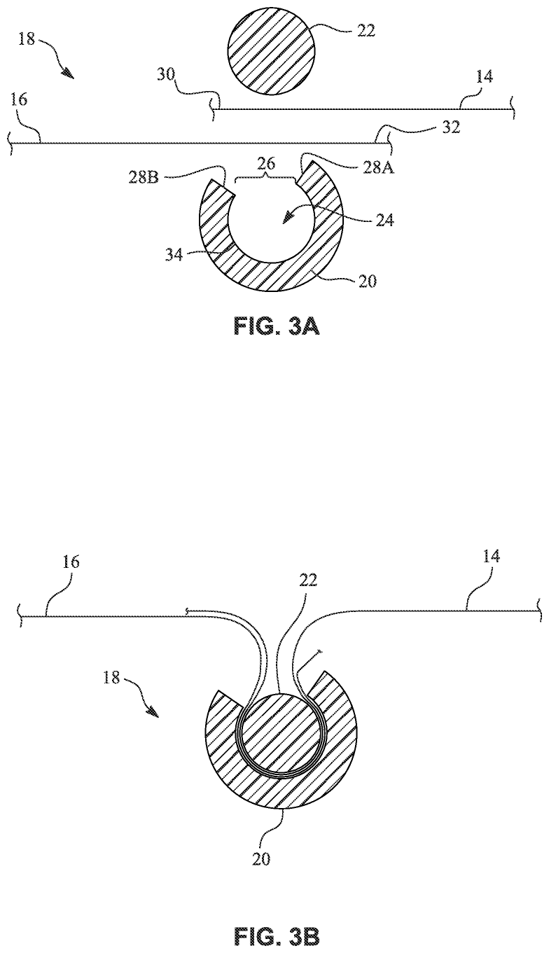

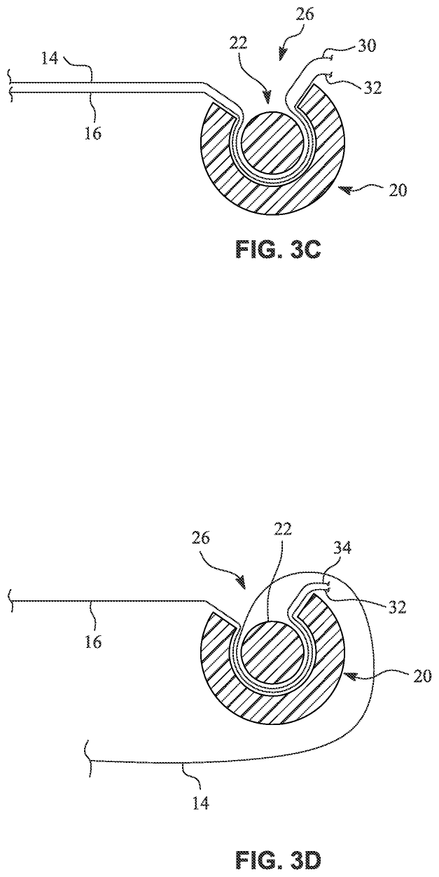

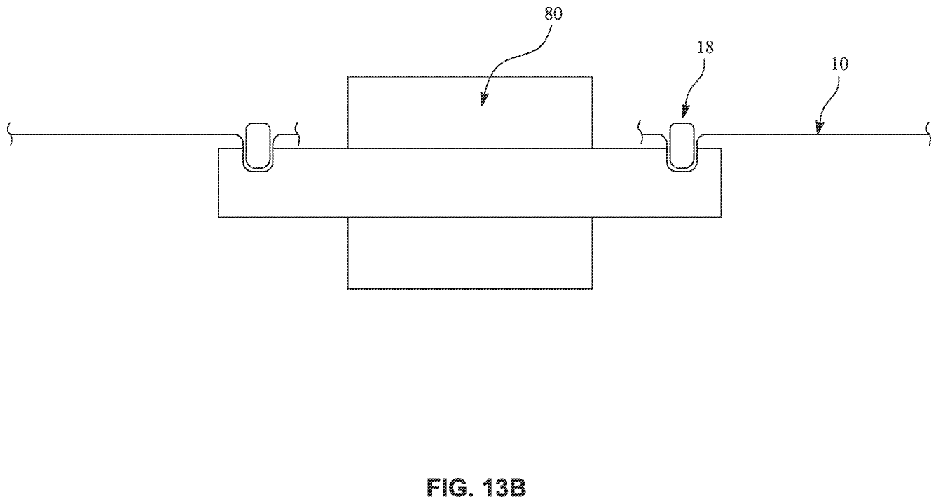

Example connector system receiving body and locking body configurations will be described in greater detail with respect to FIGS. 3-12. In addition, an example connector system configuration for attaching an accessory feature to a drape will be described with respect to FIGS. 13A and 13B. However, an example drape formed of multiple sections of drape material joined together with a connector system will first be descried with respect to FIGS. 1 and 2.

FIG. 1 is an illustration of an example medical drape 10 according to the disclosure. Drape 10 is illustrated as being implemented as a robotic surgical arm drape configured to be positioned over a surgical arm 12 of a robotic surgical system. Drape 10 can have a variety of different configurations and implementations, as discussed in greater detail herein. As shown in FIG. 1, drape 10 can be formed from at least two physically separate sections of drape material, which is illustrated as including a first section of drape material 14 and a second section of drape material 16, that are joined together to form a combined structure (e.g., unity structure, integral structure). First section of drape material 14 and second section of drape material 16 are joined together by a connector system 18. Connector system 18 can interlock portions of first section of drape material 14 and second section of drape material 16 together, forming a closed (e.g., sealed) joint line between the two sections of material.

FIG. 2 is an illustration of connector system 18 that can be used on drape 10. Connector system 18 may be formed from a receiving body 20 and a locking body 22. Receiving body 20 can define a groove 24 into which locking body 22 is inserted. As will be described, a portion of the first section of drape material 14 and the second section of drape material 16 can be positioned in and/or over groove 24 of receiving body 20. Locking body 22 can subsequently be inserted into groove 24, sandwiching the portions of the two drape materials positioned in the groove between the receiving body and the locking body. In this way, connector system 18 can join the first section of drape material 14 and the second section of drape material 16 together to form drape 10. While supplemental attachment means may also be used to join the first section of drape material 14 and the second section of drape material 16 (e.g., adhesive, stitching, heat bonding), in some examples, the two sections of drape material are joined together by connector system 18 without using any supplemental attachment means.

FIGS. 3A and 3B (collectively referred to as “FIG. 3”) are illustrations showing an example interlocking arrangement of connector system 18. FIG. 3A illustrates the first and second sections of drape material 14, 16 and locking body 22 positioned disengaged from receiving body 20. FIG. 3B illustrates a portion of the first and second sections of drape material 14, 16 inserted into receiving body 20 and locking body 22, thereby locking the inserted portions in the receiving body.

In general, receiving body 20 may define a groove 24 configured (e.g., sized and/or shaped) to receive one or more portions of material inserted therein (e.g., portions of the first and second sections of drape material 14, 16) as well as at least a portion of receiving body 20 also inserted therein. Enough of the first section of drape material 14 and the second section of drape material 16 may be positioned in groove 24 so that the corresponding section is trapped in the receiving body 20 by locking body 22, when the locking body is inserted in the receiving body. In some examples, a portion of the first section of drape material 14 and/or a portion of the second section of drape material 16 is positioned extending partially but not fully across the width of groove 24 with locking body 22 inserted into the groove. In other examples, a portion of the first section of drape material 14 and/or a portion of the second section of drape material 16 is positioned extending fully across the width of groove 24 (e.g., bridging across the groove) with locking body 22 inserted in the groove. In either case, one or both sections of drape material can overlay a portion or entirety of the width of groove 24 defined by receiving body 20, and locking body 22 can subsequently be inserted into the receiving body to trap the overlaying portions of the sections of drape material in the groove.

In the example of FIG. 3, groove 24 is illustrated as defining an opening 26 between a first edge 28A of locking body 22 and a second edge 28B of the locking body. As seen in FIG. 3A, a portion of the first section of drape material 14 is positioned overlaying an entirety of the width of groove 24 between first edge 28A and second edge 28B before positioning locking body 22 in the groove. In addition, a portion of the second section of drape material 16 is positioned overlaying the entire width of groove 24 between first edge 28A and second edge 28B before positioning locking body 22 in the groove. FIG. 3B illustrates locking body 22 subsequently inserted into the groove 24, capturing the portion of the first section of drape material 14 overlaying the groove and the portion of the second section of drape material 16 also overlaying the groove. Once locking body 22 is inserted in the groove as shown in FIG. 3B, the respective portions of drape material sections overlaying the groove may continue to overlay the groove (e.g., with terminal ends optionally extending outward beyond and edge defining the opening 26 of the groove), with locking body 22 locking both sections of drape material in the groove and thereby joining the different sections of drape material together.

With further reference to FIG. 3, the first section of drape material 14 is illustrated as defining a first terminal end 30, and the second section of drape material 16 is illustrated as defining a second terminal end 32. The first terminal end 30 of the first section of drape material 14 can be positioned extending past the second edge 28B defining the opening of groove 24 (e.g., in a widthwise direction). The second terminal end 32 of the second section of drape material 16 can be positioned extending past the first edge 28A defining the opening of groove 24 (e.g., in the widthwise direction). When so configured, the ends of the two different sections of drape material may be positioned on opposite sides of the opening 26 of groove 24, with the different sections of drape material overlapping each other. In other implementations, the different sections of drape material may not overlap each other but, instead, the first and second terminal ends 30, 32 of the two different sections of drape material may be positioned in groove 24 with locking body 22 inserted in the groove to trap the different sections of material in the groove.

FIGS. 3A and 3B illustrate the first section of drape material 14 and the second section of drape material 16 entering groove 24 from opposite sides of receiving body 20. As illustrated, for example, the first section of drape material 14 is positioned on the right side of receiving body 20 with a comparatively small portion of the drape section extending across groove 24 (e.g., on the left side of the groove). In addition, in the illustrated example, the second section of drape material 16 is positioned on the left side of receiving body 20 with only a comparatively small portion of the drape section extending across groove 24 (e.g., on the right side of the groove).

In other implementations, the first and second sections of drape material 14, 16 can enter opening 26 of groove 24 from the same direction rather than entering the groove from opposite sides of the opening. After locking body 22 is inserted into receiving body, one sheet of drape material may be wrapped around a top or bottom side of receiving body 20 and/or locking body 22 to position the different sheets of drape material on opposite sides of connector system 18. For example, FIGS. 3C and 3D illustrate an alternative arrangement where the first and second sections of drape material 14, 16 enter opening 26 of groove 24 from the same direction (e.g., from the left side of the groove in the illustrated example). The first terminal end 30 of the first section of drape material 14 and the second terminal end 32 of the second section of drape material 16 can each be positioned extending past the same edge defining the opening of groove 24 (e.g., in a widthwise direction), as illustrated in FIG. 3C, and locking body 22 inserted into receiving body 20. After inserting locking body 22 into receiving body 20, one sheet of drape material (e.g., first sheet of drape material 14) may be wrapped around a top or bottom side of receiving body 20 and/or locking body 22 as illustrated in FIG. 3D.

With further reference to FIGS. 3A and 3B, and depending on the configuration and orientation of sections of drape material relative to locking body 22, the sections of drape material may be positioned in overlapping arrangement with each other, with one sheet position on top of the other sheet. For example, receiving body 20 can have an inner surface 34 defining groove 24. Upon attaching the different sheets of drape material via connector system 18, one sheet of drape material can be positioned contacting the inner surface 34 of groove 24. A second sheet of drape material can be positioned overlaying both the groove 24 and the portion of the sheet of drape material contacting the inner surface 34 of the groove. Finally, locking body 22 can be installed on an outward facing surface (e.g., surface facing away from inner surface 34) of the second sheet of drape material.

Locking body 22 can be configured to have any desired lengthwise size and/or shape. The size and shape of locking body 22 can vary depending on the configuration of drape 10 (FIG. 1) and the surface the drape is intended to cover. In configurations where drape 10 is a flat or planar drape, connector system 18 (including receiving body 20 and locking body 22) may have a lengthwise size and/or shape configured to extend linearly across the drape (e.g., without bending or arcing). In other configurations where drape 10 is not a flat drape but instead includes an angle or curvature, connector system 18 (including receiving body 20 and locking body 22) may be shaped to match the angle or curvature of the drape.

In some configurations, at least a portion of drape 10 may surround an object to be draped. In these and other configurations, connector system 18 may extend about a perimeter of the drape (e.g., sections of different drape material that are combined together by the connector system to form the drape). For example, with further reference to FIG. 2, the first section of drape material 14 and the second section of drape material 16 may each be or include a tunnel. When so configured, receiving body 20 of connector system 18 may define a frame extending about a perimeter of the tunnel (e.g., an interior perimeter of the tunnel or an exterior perimeter of the tunnel). Locking body 22 can be inserted into the groove 24 defined by the frame, thereby forming a joint extending about the entire perimeter of the tunnel and connecting the first and second sections of drape material together. In some configurations, the perimeter frame has an open core or lumen, allowing a continuous tunnel to be formed from one drape section joined to the other drape section, including through the connector system 18 joining the two drape sections together. In other configurations, the perimeter frame may be closed or have a solid cross-sectional area.

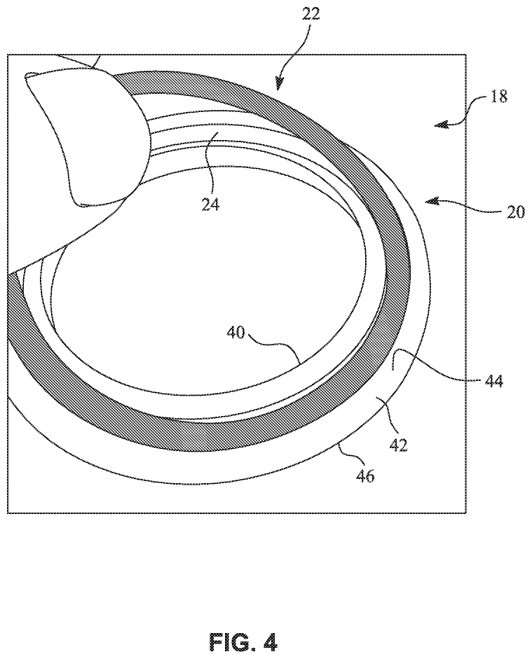

When connector system 18 (including receiving body 20 and locking body 22) are configured to extend about a perimeter of different sections of drape material, the components of the connector system may have any shape (e.g., shape in a plane perpendicular to a longitudinal axis defined by a tunnel formed by the drape). For example, receiving body 20 and/or locking body 22 may have any arcuate (e.g., circular, oval, elliptical) or polygonal (e.g., triangle, square, pentagon, hexagon, octagon) shape, or combinations of arcuate and polygonal shapes (e.g., a D-shape). While locking body 22 may typically be configured to have the same shape as receiving body 20, the two components may have different shapes provided an interlocking connection can be made between the two components. Locking body 22 may be configured to be fully received into receiving body 20 upon assembly (e.g., such that the outer-face surface of locking body 22 is flush with or recessed below the outer surface defining the groove). Alternatively, locking body 22 may be configured to extend at least partially out of receiving body 20 upon assembly (e.g., such that a portion of locking body 22 extends out of groove defined by receiving body 20).

Where the first section of drape material 14 and the second section of drape material 16 are configured with circular tunnel regions to be joined together, connector system 18 may be configured as a circular connector. FIG. 4 is a perspective view of an example configuration of connector system 18 configured as an annular connector. As shown in this example, receiving body 20 defines a circular annulus having a rim of material defining groove 24 and an inner radius devoid of material. Locking body 22 is illustrated as having a circular shape that can be positioned in groove 24 to lock sections of tunnel drape material captured between the groove and locking body.

Groove 24 can be formed in or positioned on any face of the frame surrounding the perimeter of the drape sections to be joined together by connector system 18. For example, the perimeter frame structure of receiving body 20 may define an inner face 40 (facing an interior of the frame), an outer face 42 (facing an exterior environment), a first sidewall 44 joining the inner face 40 to the outer face 42, and a second sidewall 46 joining the inner face 40 to the outer face 42 on another side of the frame. The specific number and configuration of sidewalls can vary depending on the cross-sectional shape of the frame. For example, where receiving body 20 has a circular cross-sectional shape, the inner and outer faces 40, 42, and sidewalls 44, 46, may be different locations along the continuous radius of curvature.

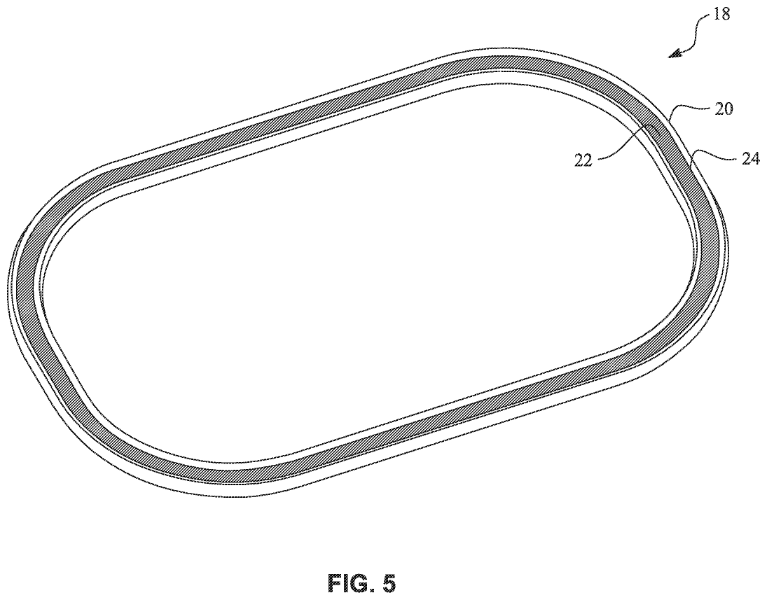

In the example of FIG. 4, groove 24 is illustrated as being provided on the first sidewall 44 of receiving body 20 (e.g., where the sidewall faces an axial direction of the tunnel sections to be joined together). FIG. 2 illustrates an alternative configuration where groove 24 is provided on the outer face 42 of receiving body 20 (e.g., where the outer face faces radially outward from the tunnel sections to be joined together). In still other examples, groove 24 may be provided on inner face 40 of receiving body 20. FIG. 5 is a perspective view of another example configuration of connector system 18 where groove 24 is provided on an axial face of the perimeter frame defining the receiving body 20 and locking body 22 is inserted in the groove. In this example, connector system 18 is illustrated as having a generally oval shape.

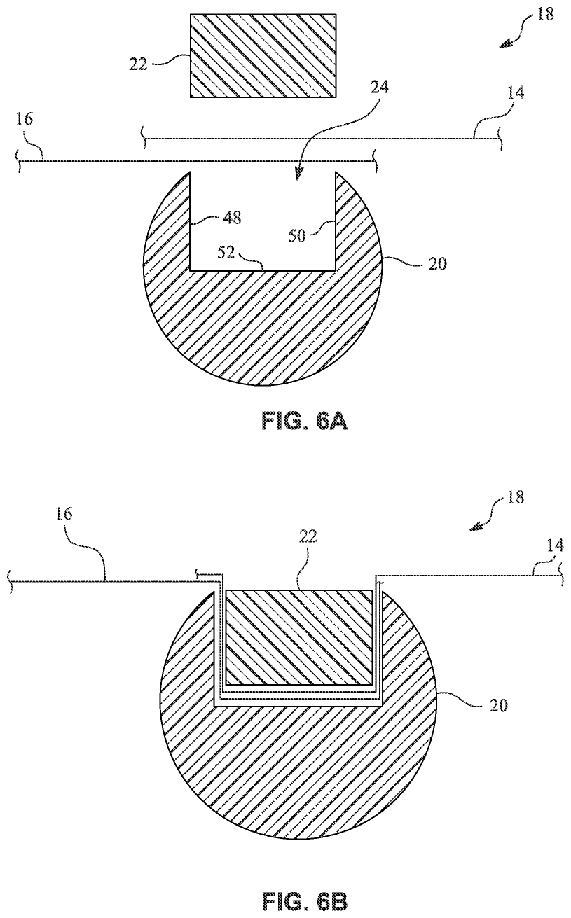

In general, groove 24 defined by receiving body 20 can be a cavity bounded by opposed sidewalls into which locking body 22 can be inserted. The sidewalls defining the groove can be curved, straight, and/or can have other configurations to help retain the locking body 22 in the groove once inserted. For example, FIGS. 6A and 6B (collectively referred to as “FIG. 6”) are illustrations showing an example interlocking arrangement of connector system 18 utilizing a groove with straight sidewalls. FIG. 6A illustrates the first and second sections of drape material 14, 16 and locking body 22 positioned disengaged from receiving body 20. FIG. 6B illustrates a portion of the first and second sections of drape material 14, 16 inserted into the groove 24 defined by the receiving body 20 and locking body 22 locking the inserted portions in the receiving body.

As shown in FIG. 6, groove 24 is bounded by a first sidewall 48, a second sidewall 50, and a bottom wall 52 connecting the first sidewall and the second sidewall. In this illustrated arrangement, the sidewalls and bottom wall are straight and extend perpendicularly relative to each other. As a result, the first sidewall 48 is parallel to the second sidewall 50, and both sidewalls intersect bottom wall 52 orthogonally.

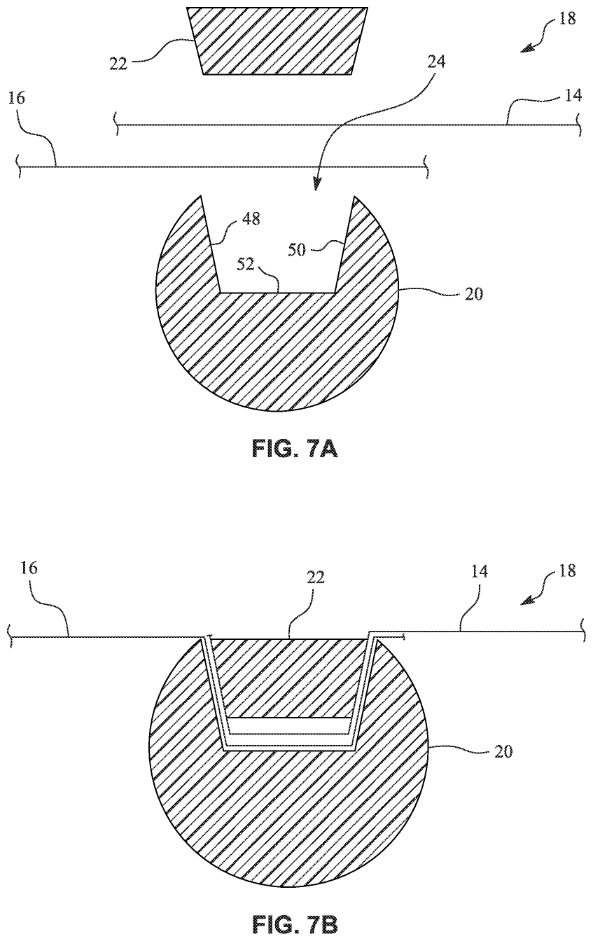

In other configurations, first sidewall 48, second sidewall 50, and/or bottom wall 52 may be angled. For example, first sidewall 48 and/or second sidewall 50 may angle inwardly relative to bottom wall 52 (e.g., forming an acute angle between the bottom wall and an intersecting sidewall) and/or angle outwardly relative to bottom wall 52 (e.g., forming an obtuse angle between the bottom wall and an intersecting sidewall). FIGS. 7A and 7B illustrate an alternative configuration of connector system 18 shown in FIGS. 6A and 6B, respectively, where the sidewalls of the groove are angled relative the bottom wall. In particular, in FIGS. 7A and 7B, first sidewall 48 and second sidewall 50 are angled outwardly relative to bottom wall 52 to form a tapered opening extending from the outermost end of the groove to the innermost end of the groove. In this case, the opening reduces in size from the outermost end of the groove (e.g., farthest away from bottom wall 52) to the innermost end of the groove (e.g., abutting bottom wall 52) but, in other configurations, may be implemented to enlarge from the outermost end of the groove to the innermost end of the groove.

As with the overall shape of connector system 18, the cross-sectional shape (across the width and height) of the connector system (receiving body 20 and/or locking body 22) may define any arcuate (e.g., circular, oval, elliptical) or polygonal (e.g., triangle, square, pentagon, hexagon, octagon) shape, or combinations of arcuate and polygonal shapes. For example, the first sidewall 48, second sidewall 50, and bottom wall 52 of groove 24 may be different locations along a continuous radius of curvature forming the groove. In some examples, receiving body 20 includes one or more features projecting (e.g., radially and/or axially) from a remainder of a sidewall and/or bottom wall into the space that would otherwise be open. Additionally or alternatively, receiving body 20 may include one or more recesses extending inwardly from a remainder of a sidewall and/or bottom wall. Configuring receiving body 20 with one or more projections and/or recesses relative to sidewall and bottom wall surfaces may be useful to create features that can help interlock with locking body 22 inserted into the receiving body.

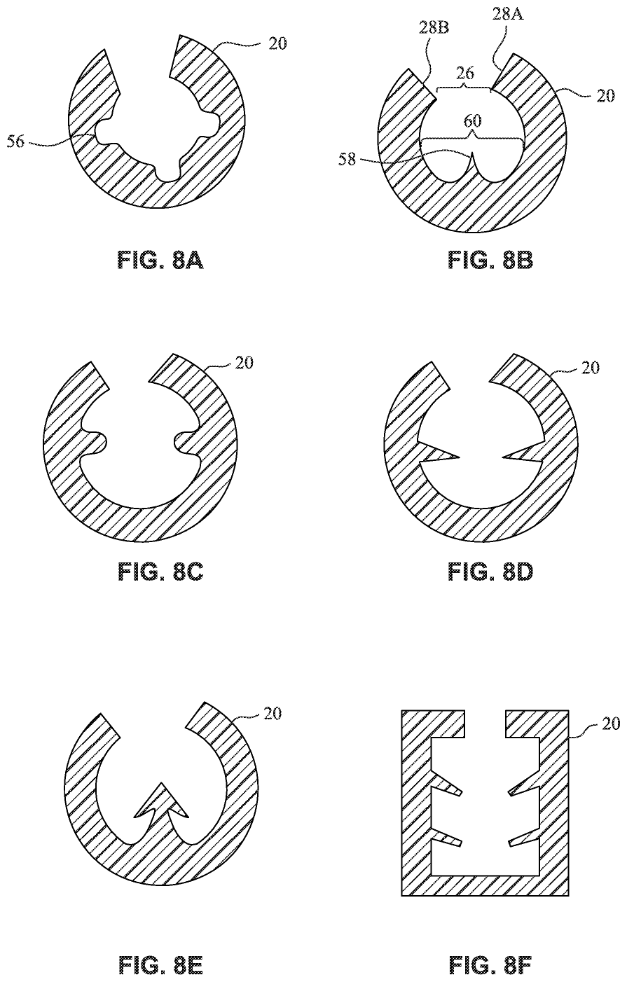

FIGS. 8A-8F are sectional views of receiving body 20 illustrating different example configurations that may be defined by the receiving cavity (e.g., groove) of the receiving body. As illustrated in FIG. 8A, receiving body 20 may include one or more recessed regions 56 that are recessed relative to a remainder of a wall surface defining the receiving cavity. As illustrated in other example configurations, such as FIG. 8B, receiving body may include one or more projections 58 that project outwardly relative to a remainder of a wall surface defined a receiving cavity. The one or more projections 58 may or may not taper to an apex, forming a barb configured to interact with and/or pierce the locking body 22, when inserted into the receiving cavity. Such recesses and projections can extend intermittently or continuously along the length of receiving body 20.

To help retain locking body 22 in receiving body 20, the opening to the groove defined by the receiving body may be narrower than a maximum width of the groove (e.g., a maximum width at a location position closer to the bottom wall of the groove than the external opening to the groove). When so configured, the sidewalls of receiving body 20 forming the groove 24 may wrap at least partially around locking body 22, when inserted into the receiving body, helping to retain the locking body in the receiving body. For example, FIGS. 8A-8F each illustrate configurations of receiving body 20 where opening 26 extending between the first edge 28A and second edge 28B is smaller than a maximum width 60 of the opening defined by groove 24 that extends across the groove.

Locking body 22 can be configured (e.g., sized and/or shaped) to fit within the configuration of groove 24 defined by receiving body 20. In some examples, locking body 22 has a complementary configuration to the configuration of groove 24. For example, locking body 22 may be sized indexed to the size of groove 24 (e.g., optionally sized slightly larger than the groove to be friction fit therein) and/or shape indexed to the shape of the groove (e.g., such that the cross-sectional shape of locking body 22 mirrors the cross-sectional shape of groove 24). In other configurations, locking body 22 may not have a complementary configuration to that of groove 24 but may be sufficiently configured and/or deformable so as to be fit into the groove.

In some configurations, locking body 22 defines groove 24 having a maximum width 60 less than 25 mm, such as less than 15 mm, less than 10 mm, less than 5 mm, or less than 3 mm. Where groove 24 of locking body 22 has straight sidewalls, opening 26 of the locking body can have the same width as maximum width 60. Where opening 26 is sized smaller than maximum width 60, opening 26 may define a width at least 0.2 mm less than maximum width 60 (that is, the difference between maximum width 60 and the width of opening 26 is at least 0.2 mm), such as at least 0.5 mm less, at least 1.0 mm less, at least 1.5 mm less, or at least 2.0 mm less. Receiving body 20 may have a size and/or shape configured to be at least partially inserted into groove 24 of locking body 22. In various examples, receiving body 20 may define a width that is the same as, less than, or greater than the width defined by opening 26 and/or maximum width 60 (e.g., in configurations where opening 26 has a different dimension than maximum width 60). For example, receiving body 20 may define a width that is slightly larger than the width defined by opening 26 of groove 24, allowing the receiving body to be press fit into the groove and subsequently retained in the groove. In some examples, receiving body 20 defines a maximum width that is less than 2.0 mm greater than the width of opening 26 (that is, the difference between the maximum width of receiving body 20 and the width of opening 26 is less than 2.0 mm), such as less than 1.5 mm, less than 1.0 mm, less than 0.5 mm, less than 0.3 mm, less than 0.2 mm, or less than 0.1 mm.

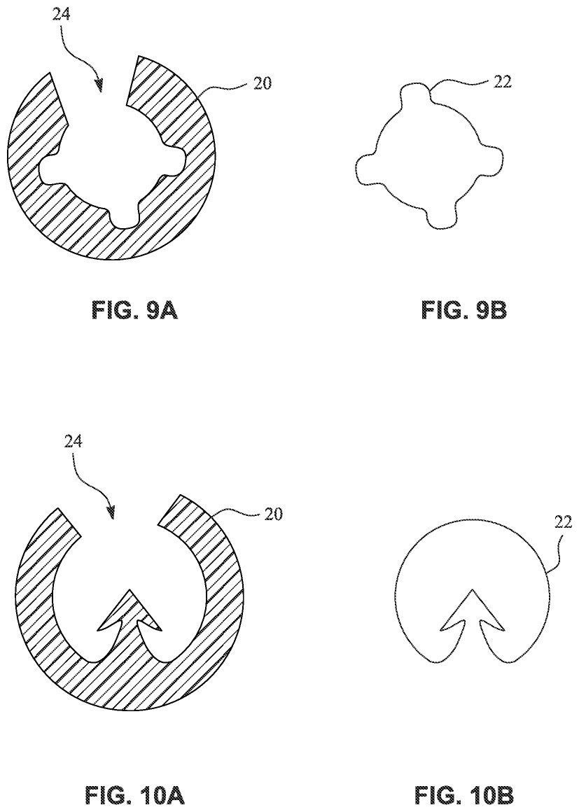

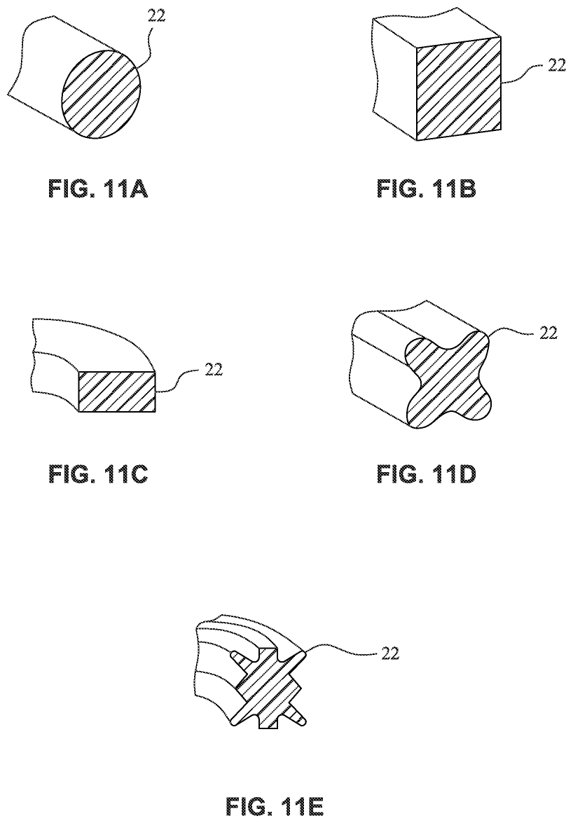

FIGS. 9A and 9B are sectional illustrations of an example configuration of receiving body 20 and locking body 22 illustrating an example complementary configuration of the groove 24 and corresponding locking body. FIGS. 10A and 10B are sectional illustrations of another example configuration of receiving body 20 and locking body 22 illustrating an example complementary configuration of the groove 24 and corresponding locking body.

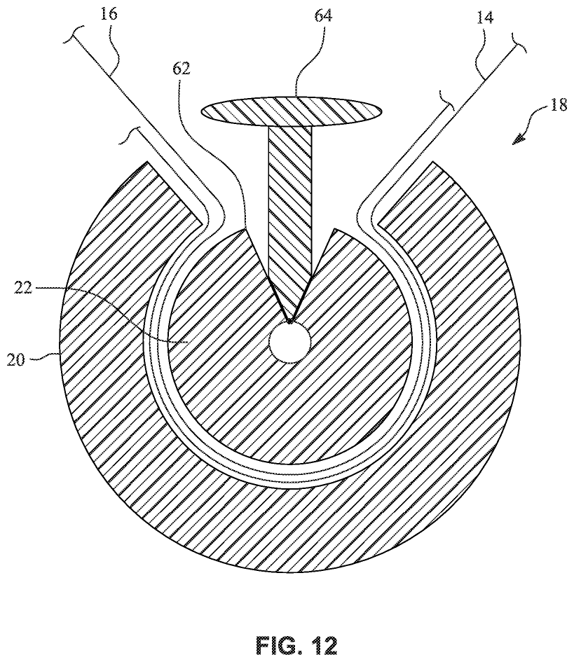

FIGS. 11A-11E are sectional views of locking body 22 illustrating example cross-sectional shapes for the locking body. FIG. 11A illustrates locking body 22 have a circular cross-sectional shape. FIG. 11B illustrates locking body 22 having a square cross-sectional shape. FIG. 11C illustrates locking body 22 having a rectangular cross-sectional shape. FIG. 11D illustrates locking body 22 having a star cross-sectional shape. FIG. 11E illustrates locking by 22 having a double star cross-sectional shape. Other cross-sectional shape configurations can be used without departing from the scope of the disclosure.

In use, locking body 22 can be inserted into receiving body 20 and retained within the receiving body to hold sections of drape material. The locking body can be inserted by the manufacture of drape 10 to provide an assembled device for sale or use. Alternatively, the locking body can be provided separated from the receiving body, and the locking body can be inserted by an end user, e.g., to allow the end user to custom build a drape from a system of multiple different drape components each having a different configuration. In some examples, to help retain locking body 22 in the receiving body 20 once inserted, locking body 22 may be expanded once inserted into the groove. For example, locking body 22 may have a split seam or other partition feature allowing the locking body to be spread apart so as to enlarge its cross-sectional size. After inserting locking body 22 into receiving body 20, the locking body may be enlarged so as to help prevent the locking body from inadvertently coming out of the receiving cavity.

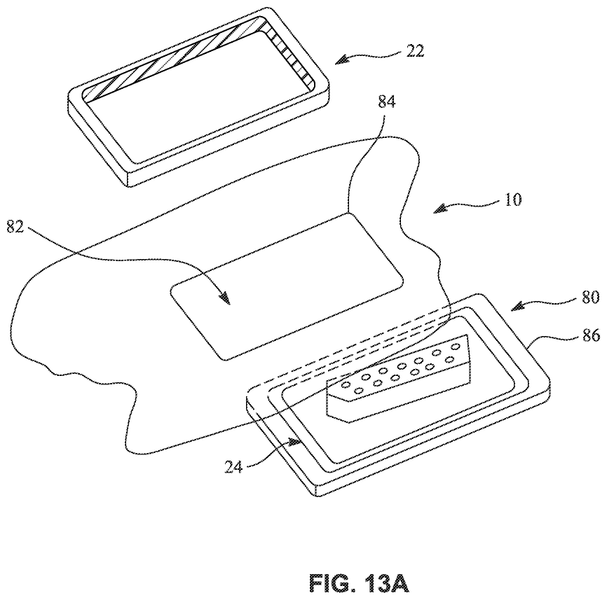

FIG. 12 is a side view of an example configuration of connector system 18 in which locking body 22 is configured to expand after being inserted into receiving body 20. In this example, locking body 22 is configured with an axial split 62. After inserting locking body 22 into receiving body 20, a partition member 64 can be inserted into the axial split so as to cause the locking body to expand, increasing the width of the locking body in the groove.

Drape 10, including first section of drape material 14 and second section of drape material 16, can be fabricated from any suitable material or combinations of materials. For example, drape 10, including the individual components thereof, may be fabricated from a polymeric material, a fiber cloth (e.g., woven or nonwoven cloth, such as cotton cloth), and/or paper. Example types of polymeric materials that may be used to fabricate drape 10 (e.g., first section of drape material 14 and/or second section of drape material 16) include, but are not limited to, polyethylene, polypropylene, polystyrene, polyurethane, other types of polymeric material, and/or combinations thereof. Drape 10 (including first section of drape material 14 and/or second section of drape material 16) may have a thickness within a range from 0.025 mm to 1.0 mm, such as from 0.1 mm to 0.5 mm, from 0.025 mm to 0.15 mm, or from 0.05 mm to 0.08 mm. First section of drape material 14 may have the same thickness of second section of drape material 16, or one section of drape material (e.g., first section of drape material 14) may be thickness than another second of drape material (e.g., second section of drape material 16).

In some implementations, first section of drape material 14 and second section of drape material 16 are each fabricated of the same type of material. For example, first section of drape material 14 and second section of drape material 16 may each be fabricated from the same type of polymeric material. In other examples, first section of drape material 14 and second section of drape material 16 may be fabricated from different materials. For example, the two different sections of drape material may be formed from different types of polymeric materials, a same type of polymeric base material having different additives or compositional characteristics, and/or different types of material types (e.g., a polymeric material and a cloth and/or paper material). In instances where different sections of drape material forming drape 10 are composed of different types of materials (e.g., each having a different chemical composition), connector system 18 may advantageously join the different material types together (e.g., without requiring chemical or thermal bonding of the different material types, which may be challenging to chemically or thermally bond together given their different material properties).

In one example, one section of drape material forming drape 10 is fabricated from a polymeric material exhibiting a higher tear resistance than a second section of drape material forming the drape. The second section of drape material may or may not also be formed of a polymeric material, such as one exhibiting a lower tear resistance than the first section of drape material. When so configured, the region of drape 10 formed of the high tear resistance polymeric material may be configured to be positioned over protrusions, active or moving elements, and/or other features being draped where there is an increased likelihood that the drape may be punctured or torn by the underlying equipment being draped. The region of drape 10 formed of the lower tear resistance polymeric material may be joined to extend away from the high tear resistance polymeric material, draping additional surfaces that do not need the same level of tear resistance. The tear resistance of different sections of drape material can be measured according to ASTM D1004-21.

Connector system 18 including the components thereof (e.g., receiving body 20 and locking body 22) can also be formed of any suitable type or types of materials. In some configurations, one of receiving body 20 and locking body 22 is relatively rigid and the other of the receiving body and locking body is relatively deformable. For example, receiving body 20 may be formed of a comparatively rigid material whereas locking body 22 may be formed of a relatively deformable material. Configuring one of the components to be relatively rigid and the other of the components to be relatively deformable may be beneficial to allow the deformable component to deform as the locking body 22 is inserted in the receiving body 20. However, configuring the other of the components to be relatively rigid can be useful to retain the deformable component, once the locking body is inserted into the receiving body. In some examples, the relatively deformable component is elastically deformable such that it substantially returns to its original shape after deformation. In still other examples, both receiving body 20 and locking body 22 may be rigid, or both may be deformable. In one example, both receiving body 20 and locking body 22 are formed of molded or extruded polymeric material. For example, both receiving body 20 and locking body 22 may be fabricated from the same polymeric material providing the same material properties (e.g., rigidity, deformability) for both components.

In one example, receiving body 20 is fabricated from a comparatively rigid material (e.g., plastic, metal). Locking body 22 in this example may be fabricated from a comparatively deformable material (e.g., elastomeric polymer) or a comparatively rigid (e.g., formed of the same material as the material forming receiving body 20). Even though receiving body 20 is fabricated from a comparatively rigid material in this example, the sidewalls defining the groove of the receiving body may or may not be sufficiently thin to allow the sidewalls to flex away from each other during insertion of the receiving body and the groove. For instance, during assembly, locking body 22 can be inserted into receiving body 20. As the locking body 22 is pushed through the opening of the groove into the receiving body 20, the sidewalls defining the groove may push apart (e.g., enlarging the opening axis and the groove) and/or locking body 22 may deform to fit within the opening of the groove. In instances where the sidewalls push outwardly to enlarge the opening as locking body 22 is inserted, the sidewalls may substantially return to their original position after the locking body is inserted (e.g., reducing the size of the opening across the groove to help retain the locking body in the groove).

Example materials that can be used for one or both of receiving body 20 and locking body 22 include flexible materials such as films, non-woven materials, elastomeric materials, foam materials, rigid components, such as molded or machined polymers or metals, and combinations thereof. In some examples, the different components of connector system 18 (receiving body 20 and locking body 22) are different colors, which may provide a visual indication and/or color change when overlapping each other, helping to indicate when the components are properly joined. For example, receiving body 20 may define a first color (e.g., yellow) and locking body 22 may define a second color (e.g., blue) which, when combined together, define a third color (e.g., green) different than either the first or the second color.

As generally discussed above, drape 10 according to disclosure can be used to drape any desired object or combination of objects, including a variety of different types of surgical equipment used in an operating environment. In one specific implementation, drape 10 may be configured to drape at least a portion of a surgical robot used during a robotically guided surgical procedure (e.g., minimally invasive procedure). In general, a surgical robot includes one or more robotic arms that are manipulable during a surgical procedure. Each robot arm may include a robotic arm base and an end effector, separated by a series of one or more linkages that are connected by a series of one or more joints. Each joint can be independently actuatable, e.g., by an independently controllable motor. The end effector of the robotic arm may include an instrument driver (alternatively referred to as “instrument drive mechanism” or “instrument device manipulator”) that incorporates electro-mechanical means for actuating a medical instrument and/or a removable or detachable medical instrument, which may be devoid of any electro-mechanical components, such as motors.

A drape according to the disclosure may be configured to drape at least a portion of the arm of the robotic surgical system. When installed, drape 10 may extend over the end effector of the robotic arm (and/or over a terminal end of the arm connected to the end effector) and extend back up the arm, away from the end effector. For example, in the configuration of FIG. 1, drape 10 is illustrated as including first section of drape material 14 defining an enclosure (e.g., a bag, which may or may not include one or more pass through apertures) positioned over the end of the arm 12 of the surgical robot and a tunnel extending along a first portion of the arm away from the enclosure. Second section of drape material 16 defines a tunnel extending along a second portion of the arm from the first section of drape material. Connector system 18 joins the tunnel of the first section of drape material 14 to the tunnel of the second section of drape material 16. When drape 10 is configured to drape an arm of a robotic surgical system, connector system 18 may provide a grasping location (e.g., a handhold) that can be grasped or pushed by a user during draping to help manipulate the drape over the arm.

Although connector system 18 has generally been described above as being used to connect one section of drape material to another section of drape material, the connector system can be used to make a variety of different connections other than connecting two sections of drape material together. For example, connector system 18 may be attached to a single section of drape material instead of multiple sections of drape material. This application may be useful to attach the comparatively rigid connector system 18 (e.g., a frame defined by receiving body 20) to the drape, e.g., providing a surface that a user can grasp to manipulate the drape when draping a piece of surgical equipment. As another example, connector system 18 may be attached to more than two section of drape material, e.g., by locking portions of three or more sections of drape material together with the connector system. Additionally or alternatively, connector system 18 may be used to connect one or more sections of drape material directly to a piece of equipment being draped and/or be used to connect an accessory component to a section of drape material.

For example, connector system 18 can be used to connect drape 10 (e.g., which may be formed of a single section of drape material or multiple sections of drape material joined together, with or without using a connector system as described herein) directly to a piece of surgical equipment to be draped (e.g., a surgical robot). In this example, the surgical equipment can include one portion of the connector system (e.g., the receiving body, the locking body) integrated with or otherwise attached to the surgical equipment. The surgical drape can include the other portion of the connector system (e.g., the receiving body, the locking body). The drape can be positioned over the portion of the surgical equipment desired to be draped, and the portion of the connector system carried by the drape engaged with the portion of the connector system carried by the surgical equipment to physically connect the drape to the surgical equipment.

In some examples where connector system 18 is used to connect drape 10 directly to a piece of surgical equipment, the connector system is attached to one or more sections of drape material and then the connector system is connected to the piece of surgical equipment. For example, connector system 18 can be attached to one or more sections of drape material as described herein (e.g., by inserting locking body 22 into receiving body 20 with one or more sections of drape material positioned between the locking body and receiving body). The connector system 18 with drape attached thereto can then be connected to the piece of surgical equipment, such as an arm of a surgical robot, to connect drape 10 to the piece of surgical equipment via the connector system. In some examples, the piece of surgical equipment (e.g., arm of a surgical robot) has one or more connecting features (e.g., a receiving cavity, detent) configured to engage connector system 18. For example, connector system 18 may be positioned at a location along the surgical equipment where the one or more connecting features are located and engaged with the one or more connecting features. The one or more connecting features provided by the piece of surgical equipment may retain connector system 18 (and drape material affixed thereto) to the piece of surgical equipment.

As another example, connector system 18 can be used to connect an accessory feature to a drape. In practice, a drape may be configured with a variety of different types of accessory features. Example accessory features include but are not limited to a fluid port, an electrical connection port, a sterile adapter (e.g., sterile robotic instrument adapter), a clip, a button, a magnet, the like, and combinations thereof. Connector system 18 can be used to connect the accessory feature to an opening in the drape configured to receive the accessory feature.

FIGS. 13A and 13B are exploded perspective and side views, respectively, of an example configuration of drape 10 (formed of one or more sections of drape material) illustrating an example configuration of a connector system connecting an accessory feature 80 to the drape. In this example, the section of drape material to which accessory feature 80 is joined defines an opening 82, which has a perimeter 84. The accessory feature 80 also defines a perimeter 86. As illustrated, at least a portion of the perimeter 84 of the opening 82 of the drape can be positioned overlaying a groove 24. In some examples, at least a portion of the perimeter 86 of the accessory feature 80 may also be positioned overlaying the groove (e.g., in in instances in which receiving body 20 and locking body 22 are physically separate from the drape and/or accessory feature). In other examples, receiving body 20 and/or locking body 22 may be integrated into (e.g., physically attached to or formed with) the drape and/or drape accessory feature. For example, the accessory feature may itself define a locking body, e.g., by providing a groove extending around a perimeter of the accessory feature. In either case, the drape and drape accessory feature can be aligned with the groove defined by the receiving body, and locking body 22 inserted into the receiving body so as to interlock the drape accessory to the drape. Any of the features and configurations discussed above as being applicable to a drape section, receiving body, and/or locking body can be integrated into the structure of drape accessory feature 80, a drape attachable thereto, and/or connector system used to attach the two components together.

While connector system 18 has generally been described as including groove 24 formed by two opposed sidewalls that mates with locking body 22, receiving body 20 may have other configurations that allow retention and interconnection with locking body 22. For example, instead of providing a groove bounded by opposed sidewalls, receiving body 20 may have a groove formed by an intersection of a planer surface and sidewall (e.g., an angle intersection between two surfaces), a V-shaped trough, or other configuration. In still other examples, such as when locking body 22 defines a ring of any cross-sectional shape configured to extend about an outer perimeter of receiving body 20, receiving body may not provide a groove but may instead provide a planar surface that the locking body contacts. In these examples, locking body 22 may be sized relative to receiving body 20 to form a tight fit against the planar surface of the receiving body, when wrapped or positioned thereabout.

In general, drape 10, including the components thereof, can be made of a sterile (and/or sterilizable) material. After manufacture but before use, drape 10 may or may not be sterilized to ensure that the drape provides a sterile barrier. For example, drape 10 (optionally including connector system 18) may be subject to a sterilization process, such as steam sterilization, dry heat sterilization, ethylene oxide gas sterilization, or radiation sterilization.

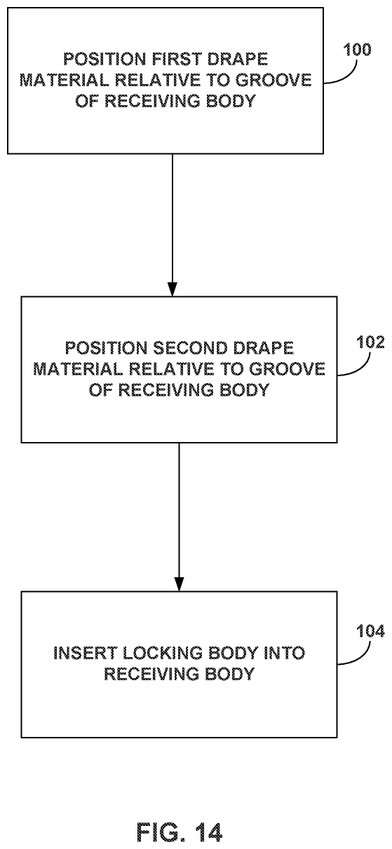

FIG. 14 is a flow diagram of an example method of attaching sections of drape material together using a connector system according to the disclosure. The example method includes positioning a portion of a first section of drape material 14 over and/or in groove 24 defined by receiving body 20 of connector system 18 (step 100 in FIG. 14). The method also includes positioning a portion of a second section of drape material 16 over and/or in the groove 24 defined by receiving body 20 of connector system 18 (step 102 in FIG. 14). In different examples, the first and second sections of drape material 14, 16 can extend over the opening to the groove 24 from the same direction or from different directions, with the remainder of the drape material extending away from the corresponding end of the drape. In either case, a portion of one section of drape material can be positioned adjacent to and/or overlapping with a portion of another section of drape material over and/in groove 24 of receiving body 20.

In some examples, the first section of drape material 14 and/or the second section of drape material 16 defines an open cavity (e.g., lumen) bounded by the drape material. For example, the first section of drape material 14 and the second section of drape material 16 may form a perimeter defining a cavity (e.g., tunnel) into which a portion of a surgical robot can be inserted (e.g., an end effector and/or a length of a robotic arm extending away from the end effector). When so configured, connector system 18 can define a frame extending partially or fully about the perimeter of the tunnel defined by the first and second sections of drape material 14, 16.

In either case, the example technique of FIG. 14 includes inserting locking body 22 into groove 24 defined by receiving body 20, thereby joining the first section of drape material 14 and the second section of drape material 16 together (step 104 in FIG. 14). Locking body 22 can have a size corresponding to the size of groove 24 defined by receiving body 20 and, in some examples, is sized slightly larger than the opening of the groove. Locking body 22 may be fabricated of a material more compressible than receiving body 20 or may have a rigidity that is substantially the same as that defined by the receiving body. In either case, locking body 22 can be inserted into receiving body 20 with a portion of the first section of drape material 14 and a portion of the second section of drape material 16 interposed between the locking body and the receiving body. Once assembled, the fit between locking body 22 and receiving body 20 may be sufficiently tight as to prohibit separation of the first section of drape material 14 from the second section of drape material 16 at connector system 18 during normal, intended use. Connector system 18 may be formed of a material and/or have a thickness such that the connector system does not substantially change size or shape after assembly whereas the one or more portions of drape material joined by the connector system may be comparatively flexible and may bend.

The first section of drape material 14 and the second section of drape material 16 can be joined together using connector system 18 during fabrication by a manufacturer or at a location where drape 10 is to be used. In either case, the resulting drape can be deployed over one or more target surfaces to be covered using the drape. In some applications, drape 10 is configured to be positioned over at least a portion of an arm of a surgical robot defining an end effector. Drape 10 can include a closed first end, an open second end, and a tunnel extending between the first end and the second end. Connector system 18 can join first and second sections of drape material 14, 16 together to define drape 10.

To drape the surgical robot in such configurations, a user may grasp portions of drape 10 and/or connector system 18 (e.g., a ring defined by the connector system) and use the connector system as a handle to guide the open end of the drape over the end effector of the surgical robot and/or to pull the drape along the arm of the surgical robot and/or end effector. The user can manipulate the open cavity defined by connector system 18 in such applications over and/or around any surface features on or extending from the end effector. The user can pull the drape up along the length of the surgical robot arm until the closed first end of the drape contacts the end of the end effector. The clinician can also pull the drape up along the length of the surgical robot arm until the second end of the drape is extended (e.g., stretched) to the length needed to drape all intended surfaces of the surgical robot. In some applications, the user engages connector system 18 with one or more connecting features defined by the surgical robot (e.g., by docking or otherwise locking a ring defined by connector system 18 at a location defined by one or more connecting features on the surgical robotic arm).

Various examples have been described. These and other examples are within the scope of the following claims.

1. A medical drape comprising:
a first section of drape material;
a second section of drape material; and
a connector system for joining the first section of drape material to the second section of drape material, the connector system comprising a receiving body defining a groove and a locking body insertable into the groove;
wherein a portion of the first section of drape material is positioned overlaying the groove, a portion of the second section of drape material is positioned overlaying the groove, and the locking body is inserted into the groove thereby locking the portion of the first section of drape material and the portion of the second section of drape material in the groove.

2. The drape of claim 1, wherein the first section of drape material has a different chemical composition than the second section of drape material.

3. The drape of claim 1, wherein the first section of drape material exhibits a higher tear resistance than the second section of drape material according to ASTM D1004-21.

4. The drape of claim 1, wherein the first section of drape material and the second section of drape material each comprise a tunnel, the receiving body defines a frame extending about a perimeter of the tunnel with the frame providing an open lumen, and the locking body extends about an exterior perimeter of the tunnel.

5. The drape of claim 4, wherein the tunnel is circular, the receiving body defines an annulus, and the locking member comprises a circular locking member.

6. The drape of claim 4, wherein the frame defines an outer face facing an external environment, an inner face facing the open lumen, a first sidewall joining the outer face to the inner face on one side of the frame, and a second sidewall joining the outer face to the inner face on another side of the fame.

7. The drape of claim 6, wherein the groove is formed in the outer face.

8. The drape of claim 6, wherein the groove is formed in the first sidewall.

9. The drape of claim 1, wherein the first section of drape material defines a bag configured to be positioned over an end of a robotic arm of a surgical robotic system.

10. The drape of claim 1, wherein:
the groove defines an opening extending between a first edge and a second edge;
the portion of the first section of drape material is positioned overlaying an entirety of the groove between the first edge and the second edge; and
the portion of the second section of drape material is positioned overlaying the entirety of the groove between the first edge and the second edge.

11. The drape of claim 1, wherein:
the portion of the first section of drape material overlaying the groove contacts an inner surface of the groove;
the portion of the second section of drape material overlaying the groove contacts the portion of the first section of drape material contacting the inner surface of the groove; and
the locking body inserted into the groove contacts the portion of the second section of drape material overlaying the groove.

12. The drape of claim 1, wherein:
the groove defines an opening extending between a first edge and a second edge;
the first section of drape material defines a first terminal edge;
the second section of drape material defines a second terminal edge;
the first terminal edge is positioned extending past the second edge of the opening; and
the second terminal edge is positioned extending past the first edge of the opening.

13. The drape of claim 1, wherein the groove is defined by a first sidewall, a second sidewall, and a bottom wall connecting the first sidewall and the second sidewall.

14. The drape of claim 13, wherein the groove is defined by a radius of curvature.

15. The drape of claim 13, wherein the receiving body further comprises at least one barb projecting into the groove, the at least one barb engaging the locking body in the groove.

16. The drape of claim 13, wherein the groove defines an opening extending between a first edge and a second edge, and the opening is narrower than a maximum width between the first sidewall and the second sidewall.

17. The drape of claim 1, wherein the receiving body is comparatively rigid and the locking body is comparatively deformable.

18. The drape of claim 1, wherein locking body has a complementary shape to a shape of the groove.

19. The drape of claim 1, wherein the receiving body is attached to one of the first section of drape material and the second section of drape material, and the locking body is attached to an other of the first section of drape material and the second section of drape material.

20. The drape of claim 1, wherein the first section of drape material, the second section of drape material, and the connector system are all sterilized.

21. A method of attaching sections of a drape material together, the method comprising:
positioning a portion of a first section of drape material in and/or over a groove defined by a receiving body of a connector system;
positioning a portion of a second section of drape material in and/or over the groove defined by the receiving body of the connector system, wherein positioning the portion of the second section of drape material in and/or over the groove comprises positioning the portion of the second section of drape material over the portion of the first section of drape material in and/or over the groove; and
inserting a locking body in the groove, thereby joining the first section of drape material and the second section of drape material.

22. The method of claim 21, wherein the first section of drape material a different chemical composition and/or a different tear resistance according to ASTM D1004-21 than the second section of drape material.

23. The method of claim 21, wherein:
the first section of drape material and the second section of drape material each comprise a tunnel and the receiving body defines a frame extending about a perimeter of the tunnel with the frame providing an open lumen;
positioning the portion of a first section of drape material in and/or over the groove and positioning the portion of the second section of drape material in and/or over the groove comprises position an end of a tunnel defined by the second section of drape material in and/or over an end of a tunnel defined by the first section of drape material, with the open lumen provided by the frame aligned with the tunnel defined with the first section of drape material and the tunnel defined by the second section of drape material; and
inserting the locking body in the groove comprises inserting the locking body extending about an exterior perimeter of the tunnel defined with the first section of drape material and/or the tunnel defined by the second section of drape material.

24. The method of claim 21, wherein the first section of drape material defines a bag configured to be positioned over an end of a robotic arm of a surgical robotic system.

25. The method of claim 21, wherein:
the groove defines an opening extending between a first edge and a second edge, and a width of the opening is narrower than a maximum width between the first sidewall and the second sidewall; and
the locking body defines a maximum width greater than the width of the opening between the first edge and a second edge but less than the maximum width between the first sidewall and the second sidewall.

26. A draped surgical robot comprising:
a surgical robot defining an arm having an end;
a first section of drape material defining an enclosure positioned over the end of the arm of the surgical robot and a tunnel extending along a first portion of the arm away from the enclosure;
a second section of drape material defining a tunnel extending along a second portion of the arm from the first section of drape material; and
a connector system joining the tunnel of the first section of drape material to the tunnel of the second section of drape material, the connector system comprising a receiving body and a locking body;
wherein the receiving body defines a frame extending about a perimeter of the tunnel of the first section of drape material and the tunnel of the second section of drape material, the frame providing an open lumen, and
the locking body is inserted into a groove of the frame, thereby trapping a portion of the first section of drape material and a portion of the second section of drape material between the groove and locking body to form a joint between the first section of drape material and the second section of drape material.

27. The draped surgical robot of claim 26, wherein the arm having the end comprises an end effector.

28. The draped surgical robot of claim 26, wherein the first section of drape material a different chemical composition and/or a different tear resistance according to ASTM D1004-21 than the second section of drape material.

29. The draped surgical robot of claim 26, wherein the frame providing the open lumen defines a circular cross-sectional shape or a D-shape.

30. The draped surgical robot of claim 26, wherein:
the groove defines an opening extending between a first edge and a second edge;
the portion of the first section of drape material is positioned overlaying an entirety of the groove between the first edge and the second edge; and
the portion of the second section of drape material is positioned overlaying the entirety of the groove between the first edge and the second edge.

31. The draped surgical robot of claim 26, wherein:
the groove defines an opening extending between a first edge and a second edge, and a width of the opening is narrower than a maximum width between the first sidewall and the second sidewall; and
the locking body defines a maximum width greater than the width of the opening between the first edge and a second edge but less than the maximum width between the first sidewall and the second sidewall.

32. The draped surgical robot of claim 26, wherein the connecting system is engaged with the surgical robot.

33. A medical drape with accessory attachment comprising:
a section of drape material having an opening formed therein, the opening defining a perimeter;
a drape accessory defining a perimeter; and
a connector system for joining the drape accessor to the section of drape material, the connector system comprising a groove extending about the perimeter of the drape accessory and a locking body insertable into the groove;
wherein at least a portion of the perimeter of the section of drape material defining the opening is positioned in and/or over the groove, and the locking body is inserted into the groove thereby locking the drape accessory in the opening.

34. The medical drape with accessory attachment of claim 33, wherein the drape accessory comprises one of a fluid port, an electrical connection port, and an adapter.
